(19) United States
(12) Patent Application Publication
(10) Pub. No.: US 2025/0375547 A1
(43) Pub. Date: Dec. 11, 2025

(54) Sterilizing Heat Infrared Emitting LED Device

(71) Applicant: Honeybee Robotics, LLC, Longmont, CO (US)

(72) Inventors: Emily P. Seto, Upland, CA (US); Kris Zacny, Altadena, CA (US); Dean Bergman, Altadena, CA (US); Hunter Williams, Denver, CO (US); Weihao Huang, Los Angeles, CA (US); Christopher James Ladd, Glendale, CA (US); Loc Troung, Los Angeles, CA (US); Thomas M. R. Thomas, Rancho Cucamonga, CA (US); Albert Ridilla, Los Angeles, CA (US); Thientu Ho, Friendswood, TX (US); Jesse Knorr, Alhambra, CA (US); Ngoc Tran, Rosemead, CA (US); Marco Garcia, Whittier, CA (US); Natasha Bouey, Pasadena, CA (US); Kathryn Bywaters, Monrovia, CA (US); Luca Frix, Los Angeles, CA (US); Robert E. May, II, Montrose, CA (US); Jack Schultz, Sierra Madre, CA (US)

(21) Appl. No.: 19/232,034

(22) Filed: Jun. 9, 2025

Related U.S. Application Data
(60) Provisional application No. 63/658,285, filed on Jun. 10, 2024.

(51) Int. Cl. A61L 2/10; A61L 2/28

(52) U.S. Cl. CPC A61L 2/10; A61L 2/28

(57) ABSTRACT

An in-situ and in-flight microbial sterilization system for eliminating microbial contamination. The system comprises a housing having at least two openings and a passageway, the passageway configured to receive an object, and at least one pneumatic dust removal system, at least one heater and at least one non-ionizing radiation emitting device, the at least one pneumatic dust removal system. The heater and non-ionizing radiation emitting device are configured to sterilize an object within the passageway.

### CROSS-REFERENCE TO RELATED APPLICATIONS

This application claims priority to U.S. Provisional Patent Application No. 63/658,285, filed on Jun. 10, 2024. The entire contents of the foregoing application are expressly incorporated herein by reference.

### BACKGROUND

The subject disclosure relates to the art of a sterilization device and, more particularly, to a bi-directional sterilization device.

Public awareness has increased of germs including the causes and risks of contamination as well as the ways in which they spread. As a result there has been increased efforts to sterilize microorganisms and purify the environment and objects. This awareness and concern of microorganisms extends to food, clothing, other textiles, personal devices and various objects.

In an effort to combat the spread of microorganisms, numerous methods have been employed. It is well established that ultraviolet (UV) radiation is effective in killing microorganisms including but not limited to surface bacteria, viruses, yeasts, molds, dust mites and flea eggs. UV radiation is used widely for sanitizing and disinfecting surfaces in various industries including healthcare (hospital, wound disinfection and healing), food processing, research laboratories, air purification systems and water purification applications. Ultraviolet radiation/light is electromagnetic radiation having a wavelength ranging from approximately ten nanometers (nm) to approximately four hundred nanometers. UV kills mainly by direct effects; that is, the photon is absorbed by an important cellular component (like DNA), which is then altered and loses its functionality.

Accordingly, while existing UV sterilization systems are suitable for their intended purposes the need for improvement remains, particularly with UV systems having the features described herein.

### BRIEF DESCRIPTION

According to one aspect of the disclosure of the sterilization device is provided. The devices includes a housing with at least two openings and a passageway, at least one pneumatic dust removal system positioned within the passageway, at least one heater positioned within the passageway, and at least one non-ionizing radiation emitting device positioned within the passageway

In addition to one or more of the features described herein, or as an alternative, further embodiments of the device may include an infrared camera.

In addition to one or more of the features described herein, or as an alternative, further embodiments of the device may include a radiation energy measurement device.

In addition to one or more of the features described herein, or as an alternative, further embodiments of the housing include the passageway connecting the at least two openings.

In addition to one or more of the features described herein, or as an alternative, further embodiments of the device may include at least one sensor disposed within the passageway, the at least one sensor configured to measure a sterilization parameter.

In addition to one or more of the features described herein, or as an alternative, further embodiments of the at least one sensor is at least one of a temperature measurement device and a radiation energy measurement device.

According to one aspect of the disclosure of the method for sterilizing an object is provided. The method includes the steps of moving an object through a passageway, flowing air onto and extracting debris from the object with a first pneumatic dust removal system within the passageway, increasing a temperature of the object to a predetermined threshold by exposing the object to a first heater within the passageway, exposing the object to a non-ionizing radiation emitting device within the passageway, and measuring at least one of a temperature or an radiation energy dose on the object within the passageway.

In addition to one or more of the features described herein, or as an alternative, further embodiments of the method may include sterilizing the object in-situ.

In addition to one or more of the features described herein, or as an alternative, further embodiments of the method may include sterilizing the object in-flight.

In addition to one or more of the features described herein, or as an alternative, further embodiments of the method may include the step activating heat shock.

In addition to one or more of the features described herein, or as an alternative, further embodiments of the method may include the step increasing the temperature gradually.

According to one aspect of the disclosure of the aerospace system is provided. The system includes a vehicle having a first housing with a first port and a sterilization device having a second port selectively coupled to the first port, the sterilization device including a passageway configured to receive an object and at least one pneumatic dust removal system, at least one heater and at least one non-ionizing radiation emitting device, the at least one pneumatic dust removal system, at least one heater and at least one non-ionizing radiation emitting device configured to sterilize a device within the passageway.

In addition to one or more of the features described herein, or as an alternative, further embodiments of the sterilization device includes a second housing having a first chamber and a second chamber with the passageway extending between the first chamber and second chamber, wherein the second port is coupled to the first chamber and the second chamber includes a third port.

In addition to one or more of the features described herein, or as an alternative, further embodiments of the sterilization device includes a temperature measurement device operably coupled to the passageway.

In addition to one or more of the features described herein, or as an alternative, further embodiments of the sterilization device includes a radiation energy measurement device.

In addition to one or more of the features described herein, or as an alternative, further embodiments of the sterilization device includes a first opening in communication with the first chamber and a second opening in communication with the second chamber.

In addition to one or more of the features described herein, or as an alternative, further embodiments of the second port is selectively coupled to the first port.

In addition to one or more of the features described herein, or as an alternative, further embodiments of the second housing is integral with the first housing.

In addition to one or more of the features described herein, or as an alternative, further embodiments of the system includes a containment device in communication with the passageway and configured to receive an object from the passageway.

The above features and advantages, and other features and advantages of the disclosure are readily apparent from the following detailed description when taken in connection with the accompanying drawings.

### BRIEF DESCRIPTION OF THE DRAWINGS

Other features, advantages and details appear, by way of example only, in the following detailed description, the detailed description referring to the drawings in which:

FIG. 1 is a schematic diagram of a sterilization device in accordance with an embodiment;

FIG. 2 is a flow diagram of a method of sterilizing an object in accordance with an embodiment; and

FIG. 3 is a perspective view of the sterilization device carried by a space vehicle.

The detailed description explains embodiments of the disclosure, together with advantages and features, by way of example with reference to the drawings.

### DETAILED DESCRIPTION

The following description is merely exemplary in nature and is not intended to limit the present disclosure, its application or uses. It should be understood that throughout the drawings, corresponding reference numerals indicate like or corresponding parts and features.

Embodiments of the present disclosure provide for a bi-directional sterilization device that sterilizes an object as it passes through the device, either forward or rearward. Embodiments of the present disclosure provide for a sterilization system that operates in environments on Earth, in space with low/micro gravity or no gravity, or in extraterrestrial environments. Further embodiments of the present disclosure provide for an object to be exposed to a combination of sterilization components such as UV wavelengths, heat, and a vacuum Still further embodiments of the present disclosure provide for a sterilization system that may be in the structure of a space vehicle.

Historically, UV lamps have been used to sterilize objects. These UV systems emitted only one wavelength (typically 254 nm). Furthermore, conventional UV sterilization devices do not include UV diodes with different wavelengths and do not combine other methods such as heat, chemicals (hydrogen peroxide), blowers and a validation system. As such, conventional UV sterilization devices do not fully sterilize the subject-object with exposing the object for undesirable extended time periods.

The existence of microorganisms is also a concern in the aerospace industry. Microbial contamination may be present on spacecraft as a result of manufacturing, assembly, and testing operations. As a result, this contamination may remain part of the mission lifecycle unless removed by surface decontamination or sterilization techniques. In the conducting of extraterrestrial sample return and life detection missions it is desired that sterilization strategies are provided for both forward and backward planetary protection. The existing sterilization technology typically used are insufficient for future sample return missions and sensitive hardware because of its bulky size which interferes with the limited space allocated on for assembly on space vehicles. The current state of-the-art for forward planetary protection re-contamination prevention has been the biobarrier. The purpose of the biobarrier was to protect other planets from Earth contamination rather than re-contamination during operations. Thus, even with the provided cleaning and sterilization procedures in place, spacecraft hardware is still susceptible to re-contamination or redistribution of organisms from a non-sterile environment to sterile hardware. Re-contamination and fallout can occur during any stage from initial assembly through rework, test, launch operation, cruise, and EDL (Entry, Descent, and Landing). After last access, any recontamination will remain present until mission end of life and can potentially interfere with functionality and life-detection instrumentation. Additionally, all sterilization systems, except a biobarrier flown, have been designed for ground support. Thus making it difficult to sterilize spacecraft hardware prior to its return to Earth.

The development and implementation of a sterilization strategy is desired to mitigate both recontamination and cross-contamination throughout the spacecraft lifecycle. The present disclosure improves upon this practice by using a combination of UV LEDs, heat, and blowers/vacuum. The UV is a “no-touch” or non-contact passive sterilization approach for sensitive hardware and a well-known sterilant often used to inactivate microorganisms, configured to emit certain wavelengths, including a combination of different wavelengths. The present disclosure may sterilize various spore-forming organisms with a combination of selective UV LED wavelengths, with or without heat and vacuum. The present disclosure provides desired in-flight microbial reduction technology that can be used to mitigate 1) launch recontamination 2) cross contamination of sample-intimate surfaces during planetary sample collection and 3) contamination of sample return canisters for example, “break-the-chain.” The present disclosure has the advantage of being able to be incorporated into space vehicles, for example the Mars Sample Return. It can be mounted on various locations in the CCRS (Capture Containment Return System) to remove contaminants from the exterior of the OS (Orbiting Sample) container. Additionally, this in-flight sterilization technology can be mounted near sample-intimate surfaces (such as drills) and irradiating and heating the surfaces to reduce cross-contamination between sample collection events on planetary bodies. For example, UV LEDs for drills will be in arranged in a 360 degree configuration to not only irradiate drill of different shapes but also, to maximize the UV exposure from different angles. The incorporation of strategically placed present invention within the spacecraft can be a low power, cost effective, and flexible solution for reducing contamination.

The present invention also provides a desired in-situ microbial reduction technology that can be used to eliminate microorganisms on objects on Earth; whether that be spacecraft hardware, medical equipment, food, personal devices, or textiles.

The sterilization device described herein provides an efficient method of removing microorganisms from an object. The microorganisms listed in Table 1 are examples of the types of microorganisms that can be found on any given object and inactivated by the disclosed sterilization device. It is to be understood that there are additional microorganisms not listed in Table 1 may also be inactivated and the claims should not be so limited. The device utilizes various combinations of sterilization technologies. Multiple UV wavelengths can be used with and without a heater device under vacuum pressure generated by a dust removal system. It should be appreciated that a single UV wavelength can also be used with and without a heater device under vacuum pressure generated by a dust removal system.

Applications of the disclosed sterilization device include, but are not limited to, hospital equipment, personal objects, clothing, textiles, spacecraft hardware, and in-flight microbial sterilization for sample intimate-associated hardware (e.g. drills, sample tubes/canisters, return to Earth sample containers). An advantage of the sterilization technology disclosed herein is its low power, low risk, non-contact passive sterilization system that may be used for many applications, all without the need for a complicated or high-power electrical system. With the incorporation of the sterilization in a space vehicle, an object can be sterilized during last access or during flight which will eliminate reduce the risk of a cleaning feed-back loop. In some embodiments, the device eliminates the concern or reduces the risk of both re-contamination and cross-contamination in-situ by sterilizing sample intimate-associated hardware between different sample collection events on various planetary bodies. This would provide planetary protection for both the target body and the Earth (in the case of sample return) without significantly altering the sample. Sterile tools for collection and storage of potential biomolecules is desired for efficient end-to-end processing as demonstrated on recent missions such as Mars2020. The disclosed sterilization device will improve spacecraft cleaning and sterilization that remain compatible with spacecraft materials and assemblies, prevention of re-contamination and cross-contamination throughout the spacecraft lifecycle. The sterilization device is also compatible with spacecraft materials and assemblies, designed to prevent recontamination and cross-contamination throughout the spacecraft lifecycle. Due to the flexibility of sterilization device, it can be installed for in-flight sterilization of the orbiting sample and for future sample return mission subsystems. The sterilization device can also be applied during sample collection events with sample tubes and or canisters.

TABLE 1

| Organism | Organism Description |
|---|---|
| Bacillus Atrophaeus | Heat indicator organism |
| Bacillus Pumilus | UV - Indicator organism |
| Bacillus Subtilus | Standard spore indicator organism |
| Bacillus SP. ATCC 29669 | Heat indicator organisms |

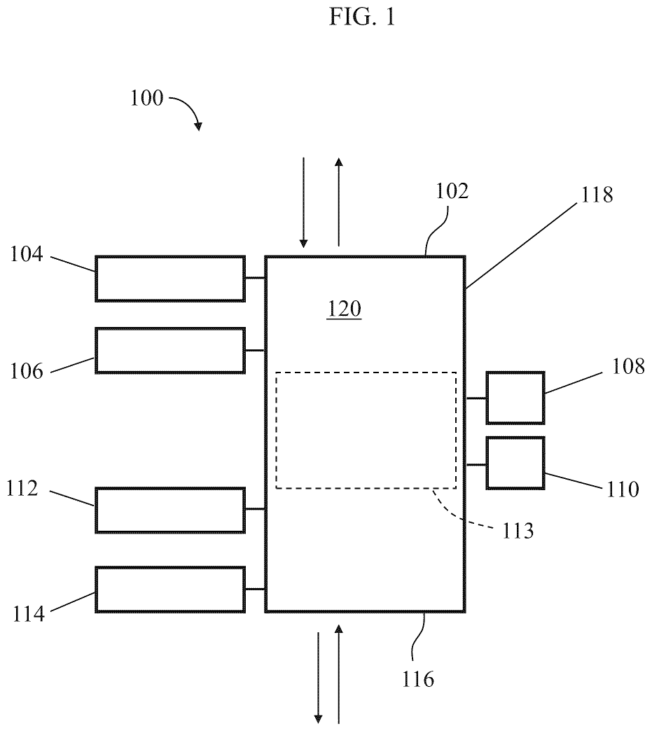

Referring now to FIG. 1, an object (not shown) enters a sterilization device 100 through a first opening 102. The object passes through the sterilization device 100 via a passageway 120. The sterilization device includes a first dust removal system 104, a first heater 106, a second dust removal system 114, and a non-ionizing radiation emitting device 112. In an embodiment, the sterilization device 100 may also include one or more sensors that measure a sterilization parameter, such as a temperature measurement device 108 (e.g. a pyrometer) or a radiation energy measurement device, 110 (e.g. a dosimeter) for example. In an embodiment, the sensors are positions in a chamber area 113 arranged between the first set of components 104, 106 and the second set of components 112, 114. The chamber area 113 provides a location or position where one or more of the sensors 108, 110 may acquire data from the target object (e.g. surface temperature) to validate whether a desired level of sterilization has occurred. It should be appreciated that in other embodiments, other sensors may be used.

The sterilization components are arranged linearly along the passageway 120. The passageway 120 connects the first opening 102 and a second opening 116. The object passes sequentially past the sterilization components. It should be appreciated that in other embodiments, the components 104, 106, 112, 114 may be arranged in a different order without deviating from the teachings herein. Depending on the object being sterilized, all, a combination, or one of the sterilization components are activated to sterilize the object. The sterilization device 100 includes sensors (not shown) that sense when an object comes into position in or near a sterilization component and when it is no longer in position in or near a sterilization component. The sensor sends a signal to a control system (not pictured) which controls the sterilization components. When an object is detected to be in position, the control system executes instructions to turn on the sterilization component. When an object is detected as no longer in position, the control system executes a different set of instruction to turn off the component.

It should be appreciated that while embodiments herein may describe the target object (e.g. a drill or tool) as entering the device 100 through opening 102 and exiting through opening 116, this is for example purposes and the claims should not be so limited. In other embodiments, the object may enter through opening 116 and exit through opening 102.

In an embodiment, the object after entering the passageway 120, the object first passes the first dust removal system 104. The dust removal system 104, 114 is compatible with the temperatures generated by the heater 106. As discussed below, there are desired temperatures an object may be heated to for a desired level of sterilization. The dust removal systems 104, 114 are able to operate in these temperature ranges. The dust removal system includes a nozzle (not shown) and the flow path geometry of the dust removal systems 104, 114 are structured to remove dust from the target object (and removing it from the passageway 120) while also keeping dust off the non-ionizing radiation emitting device 112 and heater 106 while sterilizing the object. The dust removal systems 104, 114 may utilize blowing and suctioning to dislodge debris and microorganisms off of the object and remove them from the air (or the atmosphere/vacuum of the environment where it is being used).

In an embodiment both or one of the dust removal systems 104, 112 are a pneumatic dust removal system to ensure dust removal before and/or treatment. Pneumatic dust transport is highly effective in a vacuum, allowing up to one kilogram of dust to be moved for every gram of gas. Pneumatic dust removal systems tend remove dust significantly better than brushes or other mechanical systems. Pneumatic dust removal systems also have a higher Technology Readiness Level (TRL) than electrostatic dust removal systems, making it a desired dust removal system for sterilization.

In some embodiments, the pneumatic dust removal systems convert force into potential energy, which then drives an actuator or cylinder with kinetic energy to remove air from a space. Valves are used in the pneumatic dust removal systems to control and direct the airflow, and have several different functions. To start the system, a soft start or a simple on-off valve can be used. In an embodiment, the pneumatic dust removal systems may include a flow and directional control valve, a three-way directional control, or a four-way valve. In another embodiment, the pneumatic dust removal systems may include a solenoid valve.

The pneumatic dust removal system includes an air compressor (not shown) to reduce the volume of gas in the device. This reduction of volume increases the air pressure by exciting the gas molecules. In an embodiment, a puff of gas may be introduced into the passageway 120 to create the high pressure region and static charge up occurs as a result. Once the air pressure is reduced to the desired level, one or move valves (not pictured) are opened. This action creates a vacuum force to pull the air/gas and any debris or microorganisms in the air out of the passageway 120. Depending on the environment where the object is being sterilized, for example in-flight on a space vehicle or in a hospital, it may be desired to contain the microorganism that are ejected from the passageway 120. In an embodiment, the dust removal system may include a collection tube (not pictured) that is negatively or positive charged such that it attracts the microorganisms and debris.

Following the first dust removal system 104, the object will pass by the first heater 106. A variety of heaters may be utilized, for example radiant heat, convention, conductive, or resistance heat.

In an embodiment, there is a single heater positioned at the beginning of the sequence of sterilization components. In another embodiment, there are at least two heaters, positioned at the beginning and end of the sequence. It should be noted that the sterilization device is bi-directional, meaning the object can enter the passageway 120 through either the first opening 102 or second opening 116.

The heaters are designed to apply heat to the object about a 360 degree periphery. In an another embodiment, the heaters may apply heat to the object about a periphery of less than 360 degrees. Heat is a beneficial sterilization component because the heat penetrates below surfaces and into crevices where gas cannot diffuse to. Additionally, heat transfer is not affected by lack of gravity or absence of convection as chemical processes would be. This makes heat a desired sterilization medium in the space industry as contemplated by the present application. Another advantage of this method is that objects damaged by water or steam can be sterilized provided the heat penetrates to all parts of the substance.

The heater device 106 may incorporate the following parameters: time, temperature, distance, angle, and material type of the object. The temperature applied and time the object may be subject to heat will depend on the object being sterilized. In an embodiment, the exposure time can be from 8-10 hours. In another embodiment, the exposure time can be more than 10 hours. In yet another embodiment, the exposure time can be less than 8 hours. In an embodiment, the temperature may range from 250-300 degrees Celsius. In another embodiment the temperature can be less than 250 degrees Celsius. In another embodiment the temperature can be greater than 300 degrees Celsius. The temperature measurement device 108, for example a thermal camera, is used to verify and record that the heater device has heated the object to the desired temperature.

In an embodiment, gradual heating or heat shock may be employed. With heat shock, the heater 106 is turned on and off at various intervals during the exposure time. This method shocks the microorganisms located on the object. With gradual heat, the temperature is gradually increased. Both methods assist with inactivating microorganism.

In an embodiment, a radiative heater is used. The radiative heater includes circuits configured to dissipate heat evenly about the object. Radiative heating uses electromagnetic waves to transfer energy from the infrared source to the product to be heated without heating the air between. The radiative heater produces heat waves that are absorbed through the object's body, heating it thoroughly. A radiant heater does not heat the air of the surrounding environment like other types of heaters, thereby putting less strain on the nearby sterilization components.

In another embodiment, a laser heater is used. The laser generated by the heater can be roughly 120 microns in size. In this embodiment, the laser heat ablates the surface of the object. The laser heater can include an optimum light source, fiber, and lens. In an embodiment, the light source is a laser diode.

Following the first heater 106, the object will pass by the non-ionizing radiation emitting device 112. In an embodiment the non-ionizing radiation emitting device 112 include a plurality of compact UV LEDs. Compact UV LEDs consume low power and are mechanically stable. The UV LEDs are designed to apply light to the object about a 360 degree periphery. In another embodiment, the UV LEDs may apply light to the object about a periphery of less than 360 degrees. The UV LEDs may be configured in a circular or ring shape to irradiate an object of varying shapes and sizes and to maximize the UV exposure from different angles. Based on the object being sterilized, its shape may create shielding or shadowing of other parts of itself. The circular or ring configuration allows the UV LEDs to irradiate these potentially shielded or shadowed areas.

The UV exposures may incorporate the following parameters: time, distance, angle, wavelengths, and material type. Ultraviolet radiation mainly by direct effects; that is, the photon is absorbed by an important cellular component (like DNA), which is then altered and loses its functionality. Some cells, like bacterial spores, produce a specific photoproduct (e.g., the spore photoproduct-an altered DNA base pair) when irradiated with UVC. In general, the UV-A (320-390 nm) effects cellular repair systems, respiratory membranes, and reactive oxygen species. In the UV-B (290-320 nm) killing is a mix of UV-A like effects but becomes dominated by nucleic acid effects as the wavelength decreases. In the UV-C (190-290 nm) killing closely follows the absorption spectrum of nucleic acids in the 230-290 nm region. Accordingly, most existing UV sterilization methods utilize UV-C.

The sterilization device contemplated herein utilizes UV-B and UV-C. Utilizing a larger range of wavelengths provides for combined effects of inactivation. Additionally, the parameters time and distance are the important variables in terms of viable spore reduction. Arranging the lights with closer distances and longer exposure times improve the effectiveness of sterilization. Increased UV LED time exposure increases the chances of killing all surviving microbes. The radiation energy measurement device 110, for example a dosimeter, is used to verify and record the object is being exposed to the appropriate UV wavelengths.

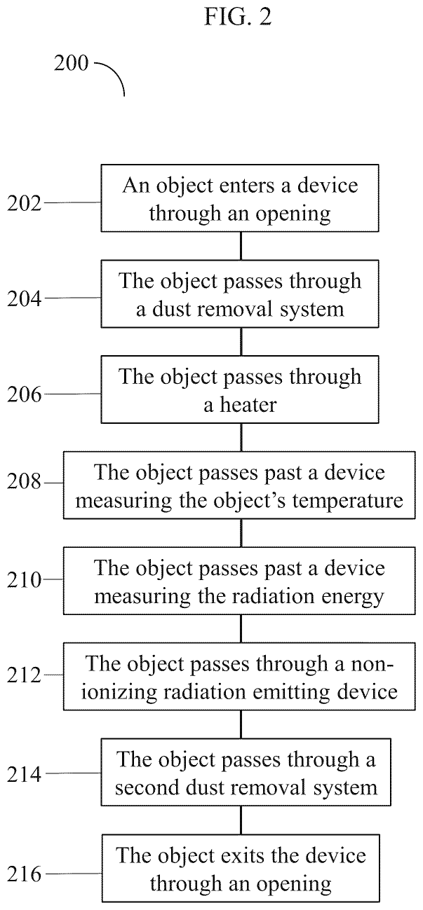

Referring now to FIG. 2, an exemplary method 200 is shown for sterilizing an object, in accordance with some disclosed embodiments. As shown in FIG. 2, method 200 may include a series of steps, some of them may be optional. As described above, the object may be subject to a combination, all, or only one of the sterilization components. For example, the object may be subject to step 204 and 212 but not step 206 or step 214. In an embodiment, the method 200 begins in in step 202, where the object enters the sterilization device. The method 200 then proceeds to step 204, where the object passes past or through a dust removal system, as described above. Next, the method 200 proceeds to step 206, where the object passes past or through a heater device, as described above. In conjunction with step 206, step 208 measures the temperature of the object. The method 200 then proceeds to step 210 in conjunction with step 212, the object passes past or through a non-ionizing radiation emitting device, as described above, and a device measures the radiation energy. In step 214, the object passes past or through a second dust removal system, as described above. In step 216, the object exits the sterilization.

As described above, the sterilization is bi-directional. Thus, step 202 or step 216 may be the first step when sterilizing the object. In an embodiment, the exemplary method may include an additional step of the object passing past or through a second heater device (not pictured).

Moreover, it will be apparent to those skilled in the art from consideration of the specification and practice of the present disclosure that various modifications and variations can be made to the disclosed systems and methods without departing from the scope of the disclosure, as claimed. Thus, it is intended that the specification and examples be considered as exemplary only, with a true scope of the present disclosure being indicated by the following claims and their equivalents.

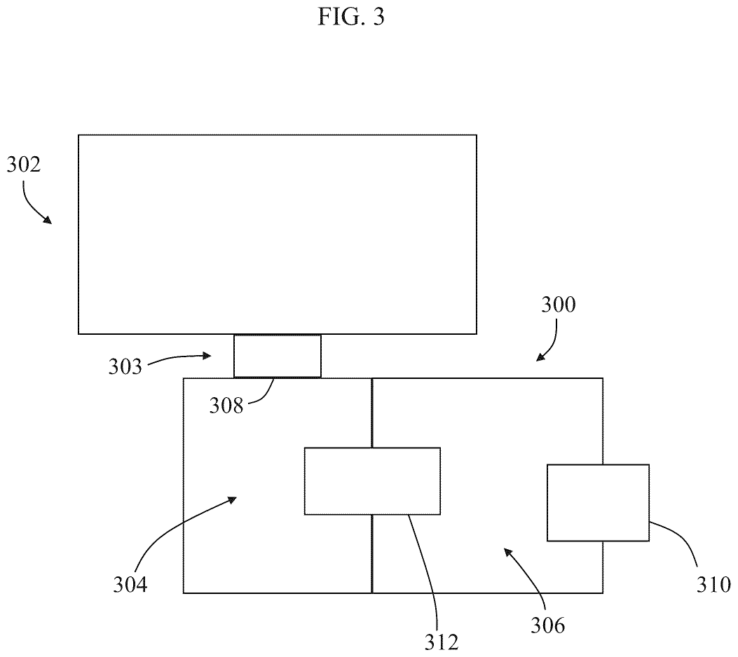

Referring now to FIG. 3, an exemplary space vehicle 302 is shown that is configured to transport a crew or a payload. The vehicle 302 includes a return module 304 that includes systems for launching the from the extraterrestrial environment. The vehicle 302 may be configured to travel back to Earth (e.g. an Earth Return Module), or dock with another spacecraft. In an embodiment, the vehicle also includes a housing having at least one port 303 configured to allow objects, such as devices, tools or samples to pass between an interior of the vehicle 302 and an external environment or another vehicle/device.

In an embodiment, a sterilization device 300 secured to the space vehicle 302 such that the object can be re-sterilized prior to deployment and sample collection and the sample collection itself can be sterilized to prevent cross contamination when returned to the vehicle 302. In an embodiment, the sterilization device 300 is integrated into the vehicle 302, or deployed/stationed on an extraterrestrial surface and is configured to selectively dock/couple with port on the vehicle.

It should be appreciated that spacecraft-based sterilization system and or local in-situ sterilization station is vital as it will reduce contamination, enhance life detection capabilities, and in turn enable scientific data return from future planetary missions.

Embodiments implement an efficient method of removing contamination from sample intimate-associated hardware. This system provides advantages in improving scientific measurement capabilities for all planetary missions by reducing the complexity, cost, mass, and time associated with implementation of a system that can mitigate microbial contamination from integration to landing on another planetary body. The sterilization device 300 provides a major improvement over conventional and costly techniques used for sterilization and a major impact for life detection. The sterilization device 300, when secured to the exemplary space vehicle 302, acts as an in-situ sterilization system that can be used to re-sterilize sample intimate-associated hardware such as drills post-launch and between sample collection events on planetary bodies and can be integrated with the following sample intimate-associated hardware to sterilize the sample.

In an embodiment, the sterilization device includes a housing having at least two portions or chambers 304, 306. The first chamber 304, sometimes referred to as a clean-zone includes a port 308 that is configured to selectively provide a pathway between the chamber 304 and the vehicle 302. In an embodiment, the port 308 may be selectively coupled to the vehicle 302. The second chamber 306, sometimes referred to as a capture and orient module is configured to release and retrieve objects from the environment through a port 310.

Movement between the first chamber 304 and the second chamber 306 occurs through a passage way within the sterilization system 312. The system 312 may be configured in the same manner as described herein with respect to FIG. 1. In other words, the system 312 includes a sequential set of devices, such as dust removal devices, heaters, non-iodizing radiation devices, that operate in the manner described herein to sterilize the target object. As discussed herein, the system 312 may operate with objects passing through to the external environment, or returning from the external environment.

In an embodiment, the system 312 is coupled to, or in communication with, a containment module 314 that receives tools, devices, objects, or sample materials retrieved or extracted from the external environment. The first chamber may include mechanisms (not shown) to move the containment module through the port 308 into the vehicle 302. In some embodiments, when the desired tools, devices, objects, or samples are retrieved, the vehicle 302 may decouple from the port 308 for a return trip to Earth or another destination.

It should be appreciated that while the description of FIG. 3 refers to the retrieval of objects from an extraterrestrial surface, this is for example purposes and the claims should not be so limited. In other embodiments, the port 310 may be configured to couple with another spacecraft or other structure for example.

It is to be understood that any structural and functional details disclosed herein are not to be interpreted as limiting the systems and methods, but rather are provided as a representative embodiment and/or arrangement for teaching one skilled in the art one or more ways to implement the methods.

It is to be further understood that like numerals in the drawings represent like elements through the several figures, and that not all components and/or steps described and illustrated with reference to the figures are required for all embodiments or arrangements.

The terms “about” and “substantially” are intended to include the degree of error associated with measurement of the particular quantity based upon the equipment available at the time of filing the application. For example, “about” and “substantially” can include a range of ±8% or 5%, or 2% of a given value.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the above disclosure has been described with reference to exemplary embodiments, it will be understood by those skilled in the art that various changes may be made and equivalents may be substituted for elements thereof without departing from its scope. In addition, many modifications may be made to adapt a particular situation or material to the teachings of the disclosure without departing from the essential scope thereof. Therefore, it is intended that the present disclosure not be limited to the particular embodiments disclosed, but will include all embodiments falling within the scope thereof.

What is claimed is:

1. A sterilization device comprising:
a housing including at least two openings and a passageway;
at least one pneumatic dust removal system positioned within the passageway;
at least one heater positioned within the passageway; and
at least one non-ionizing radiation emitting device positioned within the passageway.

2. The sterilization device according to claim 1, further comprising an infrared camera.

3. The sterilization device according to claim 1, further comprising a radiation energy measurement device.

4. The sterilization device according to claim 1, wherein the at least two openings are located at opposing ends of the sterilization device.

5. The sterilization device according to claim 4, wherein the passageway connects the at least two openings.

6. The sterilization device according to claim 1, further comprising at least one sensor disposed within the passageway, the at least one sensor configured to measure a sterilization parameter.

7. The sterilization device according to claim 6, wherein the at least one sensor is at least one of a temperature measurement device and a radiation energy measurement device.

8. A method for sterilizing an object comprising:
moving the object through a passageway;
flowing air onto and extracting debris from the object with a first pneumatic dust removal system within the passageway;
increasing a temperature of the object to a predetermined threshold by exposing the object to a first heater within the passageway;
exposing the object to a non-ionizing radiation emitting device within the passageway; and
measuring at least one of a temperature or an radiation energy dose on the object within the passageway.

9. The method of claim 8, wherein sterilizing the object includes sterilizing the object in-situ.

10. The method of claim 8, wherein sterilizing the object includes sterilizing the object in-flight.

11. The method of claim 8, wherein sterilizing the object includes activating heat shock.

12. The method of claim 8, wherein sterilizing the object includes increasing the temperature gradually.

13. An aerospace system comprising:
a vehicle having a first housing with a first port; and
a sterilization device having a second port selectively coupled to the first port, the sterilization device including a passageway configured to receive an object and at least one pneumatic dust removal system, at least one heater and at least one non-ionizing radiation emitting device, the at least one pneumatic dust removal system, at least one heater and at least one non-ionizing radiation emitting device configured to sterilize a device within the passageway.

14. The aerospace system of claim 13, wherein the sterilization device further comprises a second housing having a first chamber and a second chamber with the passageway extending between the first chamber and second chamber, wherein the second port is coupled to the first chamber and the second chamber includes a third port.

15. The aerospace system of claim 14, wherein the sterilization device further comprises a temperature measurement device operably coupled to the passageway.

16. The aerospace system of to claim 14, wherein the sterilization device further comprises a radiation energy measurement device.

17. The aerospace system of claim 14, wherein sterilization device includes a first opening in communication with the first chamber and a second opening in communication with the second chamber.

18. The aerospace system of claim 14, wherein the second port is selectively coupled to the first port.

19. The aerospace system of claim 14, wherein the second housing is integral with the first housing.

20. The aerospace system of claim 14, further comprising a containment device in communication with the passageway and configured to receive an object from the passageway.
